(19) United States
(12) Patent Application Publication
(10) Pub. No.: US 2009/0270522 A1
(43) Pub. Date: Oct. 29, 2009

(54) BLOWING AGENTS FOR POLYMERIC FOAMS

(75) Inventors: Rajiv R. Singh, Getzville, NY (US); Ian Shankland, Randolph, NJ (US); James M. Bowman, Geneva, IL (US); David J. Williams, East Amherst, NY (US)

Correspondence Address: HONEYWELL INTERNATIONAL INC.;PATENT SERVICES, 101 COLUMBIA ROAD, P O BOX 2245, MORRISTOWN, NJ, 07962-2245, US

(73) Assignee: HONEYWELL INTERNATIONAL INC., Morristown, NJ (US)

(21) Appl. No.: 12/428,543

(22) Filed: Apr. 23, 2009

Related U.S. Application Data
(60) Provisional application No. 61/047,918, filed on Apr. 25, 2008.

(51) Int. Cl. C07C 21/18; C08J 9/16; C09K 3/00

(52) U.S. Cl. 521/130; 521/131; 570/135; 252/182.15

(57) ABSTRACT

Provided are foam blowing agents comprising one or more chlorofluoroolefins selected from 1,2-dichloro-1,2-difluoroethene, 3,3-dichloro-3-fluoropropene, 2-chloro-1,1,1,3,4,4,4-heptafluoro-2-butene, and 2-chloro-1,1,1,4,4,4-hexafluoro-2-butene, as well as foams produced therefrom.

### CROSS-REFERENCE TO RELATED APPLICATIONS

This application claims the priority benefit of U.S. Provisional Application No. 61/047,918, filed Apr. 25, 2009, which is incorporated herein by reference in its entirety.

### BACKGROUND

1. Field of Invention

The present invention relates to halogenated olefin blowing agents for polymeric foams.

2. Description of the Prior Art

Production of polymeric foams typically involves the use of a chemical and/or physical blowing agent. These blowing agents serve, in part, to expand the foam via the formation of gaseous pockets within the polymeric matrix. Such blowing agents have included, for example, azo compounds, various volatile organic compounds and chlorofluorocarbons (CFCs).

Chemical blowing agents (e.g., water) generally undergo a chemical reaction during the foaming process that involves the evolution of a gas, such as nitrogen, carbon dioxide, or carbon monoxide. Physical blowing agents, on the other hand, are dissolved in a foamable polymer or polymerizable components which are capable of foaming a polymer, and then expand volumetrically (at a predetermined temperature/pressure) to contribute to the formation of the foamed structure.

Physical blowing agents are frequently used to produce foamed thermoplastic polymers, although chemical blowing agents can be used in place of or in addition to physical blowing agents in connection with thermoplastic foams. For example, it is known to use chemical blowing agent in connection with the formation of polyvinylchloride-based foams. Also it is common to use chemical blowing and/or physical blowing agents to produce foamed thermosetting polymers. Certain compounds can serve as both a chemical and a physical blowing agent.

Chlorofluorocarbons (CFCs), such as CCl₃F (CFC-11), have been used as standard blowing agents in the preparation of isocyanate-based foams, such as rigid and flexible polyurethane and polyisocyanurate foams. However, concern has increased in recent years that CFCs may damage the earth's atmosphere and climate, and accordingly, the use of certain chlorine-containing hydrocarbon compositions, such as chlorofluorocarbons (CFCs), have become disfavored because of their suspected potential to deplete the Earth's ozone. In particular, the use of CFC-11 was banned by international treaty on the ground that it was identified to have caused damages to the ozone layer upon its release into the atmosphere.

The problems associated with CFCs led to the more frequent use hydrogen-containing chlorofluoroalkanes (HCFCs). For example, CHCl₂CF₃ (HCFC-123) and CH₂ClCHClF (HCFC-141b) have been identified to have relatively short lifetimes in the atmosphere. However, while HCFCs are considered to be blowing agents relatively environmentally friendly compared to CFCs, many HCFCs still have an undesirable “Ozone Depletion Potential” (ODP). Because of the non-zero ODP, HCFCs have been targeted in recent years for eventual removal from use.

Due to a suspected association between chlorine and ODP, the industry has developed hydrogenated fluorocarbons (HFCs) as a class of non-chlorinated blowing agents. For example, CF₃CH₂CF₂H (HFC-245fa) is been widely used in insulation applications, particularly refrigerator, freezer, refrigerator/freezer and spray foam applications. However, certain HFCs possess a relatively high intrinsic thermal conductivity (i.e. poor thermal insulation), and therefore are not used as blowing agents to produce thermally insulating foams. While, certain other HFC blowing agents, such as HFC-245fa, offer improved thermal insulation, these compounds are characterized as having a relatively high global warming potential (GWP). Accordingly, it is highly desirable to use low GWP hydrofluorocarbons or other fluorinated fluids while maintaining all the other desired properties. Due to such disadvantages in connection with the use of HFCs, particularly with the aspect of rigid foam insulation, HFCs have become less desirable candidates for blowing agents in commercial foam industry.

Another known-class of blowing agents are hydrocarbon blowing agents. For example, U.S. Pat. No. 5,182,309 to Hutzen teaches the use of iso- and n-pentane in various emulsion mixtures. Another example of hydrocarbon blowing agents is cyclopentane, as taught by U.S. Pat. No. 5,096,933 to Volkert. Although many hydrocarbon blowing agents, such as cyclopentane, and isomers of pentane, are zero ozone depleting agents and exhibit very low GWP, they tend to lack adequate thermal insulation efficiency compared to foams made with, for example, a HFC-245fa blowing agent. Furthermore, hydrocarbon blowing agents are extremely flammable and often have inadequate miscibility with foamable materials, such as many of the polyester polyols commonly used to produce polyisocyanurate modified polyurethane foam. Thus, the use of these hydrocarbons frequently requires a chemical surfactant to obtain a suitable mixture, which is both undesirable and inconvenient.

U.S. Pat. No. 5,900,185 to Tapscott suggested to use certain brominated olefins as additives to decrease flammability of certain materials, including blowing agents. The additives disclosed in this patent are characterized by high efficiency and short atmospheric lifetimes, that is, they have low ODP and low GWP. While the brominated olefins described in Tapscott may have some level of effectiveness as anti-flammability agents for certain materials, these compounds also have certain disadvantages. For example, applicants have come to recognize that many of the compounds identified in Tapscott will have a relatively low efficiency if used as blowing agents, due to their relatively high molecular weight. Further, due to their relatively high boiling point, these compounds will encounter other problems when used as blowing agents. Many of the brominated olefins disclosed in Tapscott also have a high degree of bromine substitution that may render the compounds toxic and/or otherwise unsafe including the potential to develop environmentally undesirable bioaccumulation.

Accordingly, there remains a need for new compounds and compositions as attractive alternatives to conventional blowing agents. This invention satisfies this need among others.

### SUMMARY OF THE INVENTION

Applicants have recognized a need for new blowing agents that are effective and environmentally safer alternatives to the above-described blowing agents. Preferably, these alternative possess or impart properties to the foams, that are at least comparable to those associated with many of the most widely used blowing agents. Such desirable properties include vapor phase thermal conductivity (low k-factor), low- or no-toxicity, non-flammability and others. As used herein, the term “nonflammable” refers to compounds or compositions that are determined to be nonflammable in accordance with ASTM standard E-681, dated 2002, which is incorporated herein by reference. Furthermore, it is generally considered desirable for new blowing agents to be effective without major engineering changes to conventional equipment and systems used in foam preparation and formation.

Applicants have discovered unexpectedly that 1,2-dichloro-1,2-difluoroethene(cis- and trans-isomers), 3,3-dichloro-3-fluoropropene, 2-chloro-1,1,1,3,4,4,4-heptafluoro-2-butene(cis- and trans-isomers), and 2-chloro-1,1,1,4,4,4-hexafluoro-2-butene(cis- and trans-isomers) surprisingly have a low or near-zero ODP and a low GWP, demonstrate a low or no-toxicity, and are soluble in polyols. Accordingly, these compounds offer desirable alternatives to be used as blowing agents and also in other applications.

In one aspect, this invention is directed to a foamable composition comprising (a) a polymerizable material capable of forming a foam matrix; and (b) a blowing agent comprising one or more compounds selected from the group consisting of 1,2-dichloro-1,2-difluoroethene; 3,3-dichloro-3-fluoropropene; 2-chloro-1,1,1,3,4,4,4-heptafluoro-2-butene; and 2-chloro-1,1,1,4,4,4-hexafluoro-2-butene.

In another aspect, this invention is directed to a closed-cell foam comprising a cell wall comprising polyurethane or polyisocyanate and a cell volume within said cell wall, wherein said cell volume comprises at least one blowing agent selected from the group consisting of cis-1,2-dichloro-1,2-difluoroethene; trans-1,2-dichloro-1,2-difluoroethene; 3,3-dichloro-3-fluoropropene; cis-2-chloro-1,1,1,3,4,4,4-heptafluoro-2-butene; trans-2-chloro-1,1,1,3,4,4,4-heptafluoro-2-butene; cis-2-chloro-1,1,1,4,4,4-hexafluoro-2-butene; and trans-2-chloro-1,1,1,4,4,4-hexafluoro-2-butene.

In another aspect, this invention is directed to a blowing agent for a foam comprising at least one compound selected from the group consisting of cis-1,2-dichloro-1,2-difluoroethene; trans-1,2-dichloro-1,2-difluoroethene; 3,3-dichloro-3-fluoropropene; cis-2-chloro-1,1,1,3,4,4,4-heptafluoro-2-butene; trans-2-chloro-1,1,1,3,4,4,4-heptafluoro-2-butene; cis-2-chloro-1,1,1,4,4,4-hexafluoro-2-butene; and trans-2-chloro-1,1,1,4,4,4-hexafluoro-2-butene.

In yet another aspect, this invention is directed to a method for producing a foam comprising adding a blowing agent to a foamable material under conditions effective to form a foam, wherein said blowing agent comprises at least one compound selected from the group consisting of cis-1,2-dichloro-1,2-difluoroethene; trans-1,2-dichloro-1,2-difluoroethene; 3,3-dichloro-3-fluoropropene; cis-2-chloro-1,1,1,3,4,4,4-heptafluoro-2-butene; trans-2-chloro-1,1,1,3,4,4,4-heptafluoro-2-butene; cis-2-chloro-1,1,1,4,4,4-hexafluoro-2-butene; and trans-2-chloro-1,1,1,4,4,4-hexafluoro-2-butene.

### DETAILED DESCRIPTION OF PREFERRED EMBODIMENTS

Foam blowing agents of the present invention comprise one or more compounds selected from 1,2-dichloro-1,2-difluoroethene; 3,3-dichloro-3-fluoropropene; 2-chloro-1,1,1,3,4,4,4-heptafluoro-2-butene; and 2-chloro-1,1,1,4,4,4-hexafluoro-2-butene. These blowing agents have been found to be compatible with a wide variety of foaming materials and also produce a foam having desirable properties including high thermal insulation, low ozone-depletion potential, and low Global Warming Potential. Furthermore, these compounds have been found to be nonflammable and non-toxic.

Unless otherwise indicated, the chemical name for each compound refers to stereoisomers, and particularly to diastereomers, of the particular compound. For example, the term “1,2-dichloro-1,2-difluoroethene” refers to both cis and trans isomers of 1,2-dichloro-1,2-difluoroethene; the term “2-chloro-1,1,1,3,4,4,4-heptafluoro-2-butene” refers to both cis and trans isomers of 2-chloro-1,1,1,3,4,4,4-heptafluoro-2-butene; and the term “2-chloro-1,1,1,4,4,4-hexafluoro-2-butene” also includes both cis and trans isomers thereof.

The blowing agent compositions of the present invention include 1,2-dichloro-1,2-difluoroethene, 3,3-dichloro-3-fluoropropene, 2-chloro-1,1,1,3,4,4,4-heptafluoro-2-butene, and/or 2-chloro-1,1,1,4,4,4-hexafluoro-2-butene in a wide range of amounts. In certain embodiments, the blowing agent consists essentially of one or more of these compounds.

In certain embodiments, blowing agents utilized in the present invention further comprise one or more other additives. Such optional additional compounds include, but are not limited to, other compounds which also act as blowing agents (hereinafter referred to as “co-blowing agents”). The co-blowing agent can comprise a physical blowing agent, a chemical blowing agent (which preferably comprises water) or a blowing agent having a combination of physical and chemical blowing agent properties. Although it is contemplated that a wide range of co-blowing agents may be used in the present invention, the blowing agent compositions in certain preferred embodiments include one or more HFCs, more preferably one or more C₁-C₄ HFCs, and/or one or more hydrocarbons, more preferably C₄-C₆ hydrocarbons. Highly preferred HFCs include C₃ HFCs and even more preferably pentafluorinated C₃ HFCs.

For example, with respect to HFCs, the present blowing agent compositions may include one or more of difluoromethane (HFC-32), fluoroethane (HFC-161), difluoroethane (HFC-152), trifluoroethane (HFC-143), tetrafluoroethane (HFC-134), pentafluoroethane (HFC-125), pentafluoropropane (HFC-245), hexafluoropropane (HFC-236), heptafluoropropane (HFC-227ea), pentafluorobutane (HFC-365), hexafluorobutane (HFC-356) and all isomers of all such HFCs.

In certain preferred embodiments, the blowing agent compositions may include iso, normal and/or cyclo pentane for foaming thermosetting polymers. In other preferred embodiments, the blowing agent compositions may include butane or isobutane for foaming thermoplastic polymers. Other materials, such as water, CO₂, CFCs (such as trichlorofluoromethane (CFC-11) and dichlorodifluoromethane (CFC-12)), hydrochlorocarbons (such as ethyl chloride and chloropropane), HCFCs, C₁-C₅ alcohols (such as, ethanol and butanol), C₁-C₄ aldehydes, C₁-C₄ ketones, C₁-C₄ ethers (such as, dimethyl ether and diethyl ether), diethers (such as dimethoxy methane and diethoxy methane), and methyl formate, and combinations of any of the above-mentioned components, can be added. Nevertheless, such additional components are considered to be not preferred due to negative environmental impact.

In certain preferred embodiments, one or more of the following HFCs are preferred for use as co-blowing agents in the blowing agent compositions of the present invention:

1,1,1,2,2-pentafluoroethane (HFC-125)

1,1,2,2-tetrafluoroethane (HFC-134)

1,1,1,2-tetrafluoroethane (HFC-134a)

1,1-difluoroethane (HFC-152a)

1,1,1,2,3,3,3-heptafluoropropane (HFC-227ea)

1,1,1,3,3,3-hexafluoropropane (HFC-236fa)

1,1,1,3,3-pentafluoropropane (HFC-245fa) and

1,1,1,3,3-pentafluorobutane (HFC-365mfc).

The relative amount of any of the above identified co-blowing agents, as well as any additional components which may be included in present compositions, can vary widely within the broad scope of the invention and can be chosen in accordance with a particular application for the composition. In certain preferred embodiments, the co-blowing agent(s), if added, together with an sufficient amount of the compound(s) of the present invention produces a blowing agent composition which is overall nonflammable. Thus, in such embodiments, the relative amount of the co-blowing agent in comparison to the compound(s) of the present invention will depend, at least in part, upon the flammability of the co-blowing agent.

Other optional additives include surfactants, polymer modifiers, toughening agents, colorants, dyes, solubility enhancers, rheology modifiers, plasticizing agents, flammability suppressants, antibacterial agents, viscosity reduction modifiers, fillers, vapor pressure modifiers, nucleating agents, catalysts and the like.

In certain preferred embodiments, the blowing agents utilized in this invention may also include dispersing agents, cell stabilizers, surfactants and other additives. Certain surfactants can be optionally but preferably added to serve as cell stabilizers. Some representative surfactants include those sold under the names of DC-193, B-8404, and L-5340 which are, generally, polysiloxane polyoxyalkylene block co-polymers such as those disclosed in U.S. Pat. Nos. 2,834,748, 2,917,480, and 2,846,458, each of which is incorporated herein by reference. Other optional additives for the blowing agent compositions may include flame retardants such as tri(2-chloroethyl)phosphate, tri(2-chloropropyl)phosphate, tri(2,3-dibromopropyl)-phosphate, tri(1,3-dichloropropyl)phosphate, diammonium phosphate, various halogenated aromatic compounds, antimony oxide, aluminum trihydrate, polyvinyl chloride, and the like.

In certain embodiments, the co-blowing agent is selected from a group comprising, and preferably consisting essentially of HFCs, hydrocarbons and combinations of these. In certain preferred embodiments, the HFC co-blowing agent comprises a C2-C4 HFC, and even more preferably, a C3 HFC. In certain preferred embodiments, penta-fluorinated C3 HFC(s), such as HFC-245fa, is a highly preferred co-blowing agent.

In certain embodiments, the blowing agents of the present invention are used in conventional foaming equipment, such as polyurethane foaming equipment, at conventional processing conditions. The present methods include masterbatch type operations, blending type operations, third stream blowing agent addition, and blowing agent addition at the foam head. In certain embodiments, it may be desirable to utilize the blowing agent in the supercritical or near the supercritical state.

Another aspect of the present invention provides foamable compositions, preferably comprising a foamable polymer or polymerizable components and a blowing agent comprising at least one compound selected from the group of 1,2-dichloro-1,2-difluoroethene, 3,3-dichloro-3-fluoropropene, 2-chloro-1,1,1,3,4,4,4-heptafluoro-2-butene, and 2-chloro-1,1,1,4,4,4-hexafluoro-2-butene. These foams include, but are not limited to, closed cell foams, open cell foams, rigid foams, flexible foams, integeral skin foams and the like, prepared from a foamable polymer or polymerizable components. In certain preferred embodiments, the foam comprises a thermoplastic polymer. In other preferred embodiments, the foam comprises a thermosetting polymer.

Foams of the present invention may offer low or near-zero ODP and low GWP, that are associated with the blowing agents used therein. It is also believed that another advantage for the foams, particularly a foamed thermosetting polymer such as polyurethane foams, is the ability to achieve an exceptional thermal performance, as being measured by the K-factor or lambda, particularly and preferably under low temperature conditions. It is also contemplated that the foams of the present invention exhibit improved improved mechanical properties, such as dimensional stability, compressive strength, aging of thermal insulation properties. For example, the foams of the present invention may have superior compressive strengths compared to that of a foam currently commercially produced under substantially the same conditions.

Applicants believe that the foams of the present invention, particularly foamed thermosetting polymers, may be used in a wide variety of applications. In certain preferred embodiments, the foams of the present invention are appliance foams, such as refrigerator foams, freezer foams, refrigerator/freezer foams and panel foams.

In certain preferred embodiments, the foamable compositions comprise a foamable polymer or polymerizable component(s) capable of forming a thermosetting polymer upon reaction. These foamable compositions are also herein identified as thermosetting compositions. Examples of the thermosetting compositions include polyurethane and polyisocyanurate foam compositions, and also phenolic foam compositions (e.g., phenol formaldehyde based foams). Alternatively, formaldehyde-free foamable and curable polyacid/polyol polymers such as those disclosed in U.S. Pat. No. 6,221,973, which is incorporated herein by reference, can be used as thermosetting polymers with the blowing method of this invention. The reaction and foaming process for producing foamed thermosetting polymer may be enhanced through the use of various additives such as an accelerating agent (also referred to as “catalyst”) and surfactant materials that serve to control and adjust cell size and to stabilize the foam structure during formation. Furthermore, it is contemplated that one or more co-blowing agents may also be incorporated into the foamable composition of the present invention.

In certain embodiments, the foamable thermosetting compositions comprise a blowing agent of the present invention, and one or more of polymerizable components which are capable of reacting with one another and/or foaming under the proper conditions to produce a foamed polymeric matrix or cellular structure.

In certain preferred embodiments, the foamable composition comprises a foamable polymer or one or more polymerizable components that are capable of foaming a thermoplastic polymer (and/or resin). These foamable compositions are also herein identified as thermoplastic compositions. Examples of components for the thermoplastic compositions include polyolefins, such as monovinyl aromatic compounds (for example, polystyrene) having the formula Ar—CH═CH₂. Other examples of polyolefin resins include the various ethylene resins, such as polyethylene and ethylene copolymers, polypropylene (PP) and polyethyleneterepthalate (PET). In certain embodiments, the thermoplastic composition is an extrudable foamable composition.

In general, the methods of the present invention for producing a foamed polymeric matrix require incorporating the blowing agent above into a foamable composition and then foaming the foamable composition, for example by heating said composition. The method or process is preferably carried out by a step or series of steps, which result in volumetric expansion of the blowing agent. Further, conventional systems and devices for incorporation of blowing agent and for foaming are readily adaptable for use for the present invention. In fact, it is believed that one advantage of the present invention is that the improved blowing agent is generally compatible with existing foaming methods and systems. Thus, it will be appreciated by those skilled in the art that the present invention provides methods and systems for foaming many types of foams.

In certain preferred embodiments involving the production of a foam using a foamable thermosetting compositions, the method involves an exothermic reaction with one another during the foaming process. In highly preferred embodiment, such exothermic reaction(s) among the polymerizable components generate heat for use in heating the foamable composition to a temperature above the boiling point of the blowing agent used therein.

In other preferred embodiments, the polymerizable components in the foamable thermosetting compositions have an endothermic reaction with one another during the foaming process. In highly preferred embodiment, the progress of the endothermic reaction(s) among the polymerizable components also at least partially relies on the heat used to increase the temperature of the foamable composition to above the blowing agent's boiling point.

In certain preferred embodiments, the methods for producing a thermosetting polymer use various additives, particularly an accelerating agent (i.e. catalyst), to enhance the foaming process or reaction therein. All suitable accelerating agents known in the art can be used in connection with the methods of the present invention. In certain preferred embodiments, the methods for producing foamed thermosetting polymers use surfactant materials to control and adjust cell size and to stabilize the foam structure during the forming process. All suitable surfactants known in the art can be used in connection with the methods of the present invention.

It is contemplated that the methods generally comprise preparing polyurethane or polyisocyanurate foams by combining an isocyanate, a polyol or mixture of polyols, a blowing agent or mixture of blowing agents comprising one or more of the present compositions, and other materials such as catalysts, surfactants, and optionally, flame retardants, colorants, or other additives.

It is convenient in many applications to provide the components for polyurethane or polyisocyanurate foams in pre-blended compositions. Most typically, the foam composition is pre-blended into two components. The first component (commonly referred to as the “A” component) comprises the blowing agent, isocyanate and optionally certain surfactants, while the second component (commonly referred to as the “B” component) comprises polyol or polyol mixture, surfactant, catalysts, blowing agents, flame retardant, and other isocyanate reactive components. Accordingly, the foams are readily prepared by bringing together the A and B components either by hand or by machine mix techniques to form blocks, slabs, laminates, pour-in-place panels and other items, spray applied foams, froths, and the like. Optionally, other ingredients such as fire retardants, colorants, auxiliary blowing agents, and even other polyols can be added as additional stream(s) to the mix head or reaction site. Most preferably, however, they are all incorporated into the B-component.

The present methods and systems also include forming a one-component foam, preferably polyurethane foam, containing a blowing agent in accordance with the present invention. In certain preferred embodiments, a portion of the blowing agent is contained in the foam forming agent, preferably by being dissolved in a forming agent which is liquid at the pressure within the container, a second portion of the blowing agent which exists as a separate gas phase. In such systems, the contained/dissolved blowing agent performs, in large part, to cause the expansion of the foam, and the separate gas phase operates to impart propulsive force to the foam forming agent. Such one-component systems are typically and preferably packaged in a container, such as an aerosol type can, and the blowing agent of the present invention thus preferably provides for expansion of the foam and/or the energy to transport the foam/foamable material from the package, and preferably both. In certain embodiments, such systems and methods comprise charging the package with a fully formulated system (preferably isocyanate/polyol system) and incorporating a gaseous blowing agent of the present invention into the package, preferably an aerosol type can.

In certain preferred embodiments, the methods of the present invention for producing a thermoplastic foam generally comprise introducing a blowing agent in accordance with the present invention into a foamable thermoplastic material, preferably a thermoplastic polymer such as polyolefin, and then subjecting the thermoplastic material to conditions effective to cause foaming.

In certain highly preferred embodiments, the methods for producing foamed thermoplastic polymers use at least in part a melt processing to produce the heat needed for heating the foamable thermoplastic composition to a temperature above the boiling point of the blowing agent used therein.

In other preferred embodiments, the heat used for heating the foamable thermoplastic composition to a temperature above the boiling point of the blowing agent is provided at least in part by mechanically working the polymer. In certain highly preferred embodiments, the melt process used in foaming a thermoplastic material is an extrusion process. In such process, the method to foam the thermoplastic material may include the steps such as introducing a blowing agent into a screw extruder containing the thermoplastic material, lowering the pressure on the thermoplastic material and thereby causing volumetric expansion of the blowing agent.

In addition, it will be appreciated by those skilled in the art that the order and manner in which the blowing agent of the present invention is formed and/or added to the foamable composition does not generally affect the operability of the present invention. For example, in extrusion processes, the various components of the blowing agent, and even the components of the foamable composition, do not need to be mixed prior to the introduction to the extrusion equipment, and do not need to be added at the same location in the extrusion equipment.

Moreover, for the methods using extrusion processes, the blowing agent can be introduced either directly or as part of a premix before being added to the foamable composition. In certain embodiments, one or more components of the blowing agent may be introduced at first location in the extruder, which is upstream of the addition place for other component(s) of the blowing agent. And in other embodiments, the components of the blowing agent are mixed in advance before the introduction into the foamable composition.

What is claimed is:

1. A foamable composition comprising:
a. a polymerizable material capable of forming a foam matrix; and
b. a blowing agent comprising one or more compounds selected from the group consisting of 1,2-dichloro-1,2-difluoroethene; 3,3-dichloro-3-fluoropropene; 2-chloro-1,1,1,3,4,4,4-heptafluoro-2-butene; and 2-chloro-1,1,1,4,4,4-hexafluoro-2-butene.

2. The foamable composition of claim 1 wherein said 1,2-dichloro-1,2-difluoroethene consists of cis- and/or trans-isomers, said 2-chloro-1,1,1,3,4,4,4-heptafluoro-2-butene consists of cis- and/or trans-isomers, and said 2-chloro-1,1,1,4,4,4-hexafluoro-2-butene consists of cis- and/or trans-isomers.

3. The foamable composition of claim 2 wherein a blowing agent consists essentially of a compound selected from the group consisting of cis-1,2-dichloro-1,2-difluoroethene; trans-1,2-dichloro-1,2-difluoroethene; 3,3-dichloro-3-fluoropropene; cis-2-chloro-1,1,1,3,4,4,4-heptafluoro-2-butene; trans-2-chloro-1,1,1,3,4,4,4-heptafluoro-2-butene; cis-2-chloro-1,1,1,4,4,4-hexafluoro-2-butene; trans-2-chloro-1,1,1,4,4,4-hexafluoro-2-butene; and combinations thereof.

4. The foamable composition of claim 1 wherein said polymerizable material comprises a polyurethane, polyisocyanate, phenol, or precursor thereof.

5. The foamable composition of claim 1 further comprising at least one co-blowing agent selected from the group consisting of water, CO₂, methyl formate, trans-1,2,-dichloroethylene, C₁-C₄ hydrofluorocarbons, and C₄-C₆ hydrocarbons.

6. The foamable composition of claim 5 wherein said co-blowing agent is selected from the group consisting of 1,1,1,2,2-pentafluoroethane (HFC-125); 1,1,2,2-tetrafluoroethane (HFC-134); 1,1,1,2-tetrafluoroethane (HFC-134a); 1,1-difluoroethane (HFC-152a); 1,1,1,2,3,3,3-heptafluoropropane (HFC-227ea); 1,1,1,3,3,3-hexafluoropropane (HFC-236fa); 1,1,1,3,3-pentafluoropropane (HFC-245fa); and 1,1,1,3,3-pentafluorobutane (HFC-365mfc).

7. The foamable composition of claim 1 wherein said polymerizable material is a preblend consisting essentially of a first component comprising an isocyanate and a second component comprising a polyol or polyol mixture.

8. A closed-cell foam comprising a cell wall comprising a phenolic foam, polyurethane, or polyisocyanate and a cell volume within said cell wall, wherein said cell volume comprises at least one blowing agent selected from the group consisting of cis-1,2-dichloro-1,2-difluoroethene; trans-1,2-dichloro-1,2-difluoroethene; 3,3-dichloro-3-fluoropropene; cis-2-chloro-1,1,1,3,4,4,4-heptafluoro-2-butene; trans-2-chloro-1,1,1,3,4,4,4-heptafluoro-2-butene; cis-2-chloro-1,1,1,4,4,4-hexafluoro-2-butene; and trans-2-chloro-1,1,1,4,4,4-hexafluoro-2-butene.

9. A blowing agent for a foam comprising at least one compound selected from the group consisting of cis-1,2-dichloro-1,2-difluoroethene; trans-1,2-dichloro-1,2-difluoroethene; 3,3-dichloro-3-fluoropropene; cis-2-chloro-1,1,1,3,4,4,4-heptafluoro-2-butene; trans-2-chloro-1,1,1,3,4,4,4-heptafluoro-2-butene; cis-2-chloro-1,1,1,4,4,4-hexafluoro-2-butene; and trans-2-chloro-1,1,1,4,4,4-hexafluoro-2-butene.

10. The blowing agent of claim 9 further comprising a co-blowing agent selected from the group consisting of water, CO₂, C₁-C₄ hydrofluorocarbons, and C₄-C₆ hydrocarbons.

11. The blowing agent of claim 9 further comprising a co-blowing agent selected from the group consisting of 1,1,1,2,2-pentafluoroethane (HFC-125); 1,1,2,2-tetrafluoroethane (HFC-134); 1,1,1,2-tetrafluoroethane (HFC-134a); 1,1-difluoroethane (HFC-152a); 1,1,1,2,3,3,3-heptafluoropropane (HFC-227ea); 1,1,1,3,3,3-hexafluoropropane (HFC-236fa); 1,1,1,3,3-pentafluoropropane (HFC-245fa); and 1,1,1,3,3-pentafluorobutane (HFC-365mfc).

12. A method for producing a foam comprising adding a blowing agent to a foamable material under conditions effective to form a foam, wherein said blowing agent comprises at least one compound selected from the group consisting of cis-1,2-dichloro-1,2-difluoroethene; trans-1,2-dichloro-1,2-difluoroethene; 3,3-dichloro-3-fluoropropene; cis-2-chloro-1,1,1,3,4,4,4-heptafluoro-2-butene; trans-2-chloro-1,1,1,3,4,4,4-heptafluoro-2-butene; cis-2-chloro-1,1,1,4,4,4-hexafluoro-2-butene; and trans-2-chloro-1,1,1,4,4,4-hexafluoro-2-butene.

13. The method of claim 12 wherein said conditions involve chemically reacting a polyisocyanate with a polyol.

14. The method of claim 12 wherein said foamable material is a thermoplastic polymer.

15. The method of claim 12 wherein said thermoplastic polymer is selected from the group consisting of monovinyl aromatic compounds, ethylene resins, and polyethyleneterepthalate.

16. The method of claim 12 wherein said thermoplastic polymer is polystyrene.

17. The method of claim 12 wherein said thermoplastic polymer is selected from the group consisting of polyethylene and polypropylene.

18. The method of claim 12 wherein said foamable material is a thermosetting polymer.

19. The method of claim 12 wherein said foam is a closed-cell foam.

20. The method of claim 12 wherein said foam is an open-cell foam.
